(19) United States
(12) Patent Application Publication
(10) Pub. No.: US 2006/0292564 A1
(43) Pub. Date: Dec. 28, 2006

(54) Method and nucleic acids for the analysis of breast cell proliferative disorders

(75) Inventor: Sabine Maier, Berlin (DE)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics AG, Berlin (DE)

(21) Appl. No.: 10/526,108

(22) PCT Filed: Jul. 18, 2003
(86) PCT No.: PCT/EP03/07827

§ 371 Date: May 10, 2006

(30) Foreign Application Priority Data
Aug. 27, 2002 (DE) 10239313.3
Nov. 26, 2002 (DE) 10255104.9

(51) Int. Cl. C12Q 1/68; C12M 1/34

(52) U.S. Cl. 435/6; 435/287.2

(57) ABSTRACT

The present invention relates to modified and genomic sequences, to oligonucleotides and/or PNA-oligomers for detecting the cytosine methylation state of genomic DNA, as well as to a method for ascertaining genetic and/or epigenetic parameters of genes for use in the differentiation, diagnosis, treatment and/or monitoring of breast cell proliferative disorders, or the predisposition to breast cell proliferative disorders.

### FIELD OF THE INVENTION

Breast cancer is currently the second most common type of cancer amongst women. In 2001 over 190,000 new cases of invasive breast cancer and over 47,000 additional cases of in situ breast cancer were diagnosed. Incidence and death rates increase with age, for the period 1994-1998 the incidence of breast cancer amongst women between the ages of 20 and 24 was only 1.5 per 100,000 population. However the risk increases to 489.7 within the age group 75-79. Mortality rates have decreased by approximately 5% over the last decade and factors affecting 5 year survival rates include age, stage of cancer, socioeconomic factors and race.

Breast cancer is defined as the uncontrolled proliferation of cells within breasts tissues. Breasts are comprised of 15 to 20 lobes joined together by ducts. Cancer arises most commonly in the duct, but is also found in the lobes with the rarest type of cancer termed inflammatory breast cancer.

It will be appreciated by those skilled in the art that there exists a continuing need to improve methods of early detection, classification and treatment of breast cancers. In contrast to the detection of some other common cancers such as cervical and dermal there are inherent difficulties in classifying and detecting breast cancers.

The first step of any treatment is the assessment of the patient's condition comparative to defined classifications of the disease. However the value of such a system is inherently dependant upon the quality of the classification. Breast cancers are staged according to their size, location and occurrence of metastasis. Methods of treatment include the use of surgery, radiation therapy, chemotherapy and hormone therapy, which are also used as adjuvant therapies to surgery.

Predictors currently used in the assessment of breast tumours (e.g. histological analysis, estrogen receptor markers) often fail to correctly predict or classify tumour development and behaviour. Therefore, patient response to treatment and prediction of overall outcome is often not accurately predictable. The continued development of breast cancer analysis techniques is currently focused upon the investigation molecular biological markers.

The development of molecular biological markers as an alternative to traditional histopathological analysis has to date focused on the analysis of single nucleotide polymorphisms and single genes, such as BRCA1 and BRCA 2. Furthermore, in addition to oncogene mutations gene amplification, and loss of heterozygosity in invasive breast cancer (Callahan, et al., 1992; Cheickh, et al., 1992; Chen, et al, 1992; and, Lippman, et al, 1990) have also been assessed. More recently, the use of microarry technology has allowed the concurrent analysis of multiple genes as well as genetic expression profiling by analysis of RNA and proteins. The analysis of multiple loci in order to predict breast cancer risks in populations was discussed by Pharoah et. al. ‘Polygenic susceptibility to breast cancer and implications for prevention’ Nat Genet. 2002 May;31(1):33-6. Furthermore, Friend et. al. (‘Gene expression profiling predicts clinical outcome of breast cancer’ Nature 415, 530-536 (2002)) used gene expression profiling to predict the outcome of treatment in breast cancer patients, their methods may be used to enable improved post surgery treatment decisions.

However, as hereditary breast cancers only account for 5% to 10% of cases it is likely that epigenetic mechanisms, as well as hereditary mutations and environmental factors influence the development of breast cancers.

The levels of observation that have been studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

DNA methylation plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing since 5-methylcytosine has the same base pairing behaviour as cytosine. Moreover, the epigenetic information carried by 5-methylcytosine is completely lost during PCR amplification.

A relatively new and currently the most frequently used method for analyzing DNA for 5-methylcytosine is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its hybridisation behaviour, can now be detected as the only remaining cytosine using “normal” molecular biological techniques, for example, by amplification and hybridisation or sequencing. All of these techniques are based on base pairing which can now be fully exploited. In terms of sensitivity, the prior art is defined by a method which encloses the DNA to be analysed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. 1996 Dec. 15;24(24):5064-6). Using this method, it is possible to analyse individual cells, which illustrates the potential of the method. However, currently only individual regions of a length of up to approximately 3000 base pairs are analysed, a global analysis of cells for thousands of possible methylation events is not possible. However, this method cannot reliably analyse very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 March-April;5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. 1997 November;17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun. 15;25(12):2529-31, WO 95/00669) or by enzymatic digestion (Xiong Z, Laird P W. COBRA: a sensitive and quantitative DNA methylation assay. Nucleic Acids Res. 1997 Jun. 15;25(12):2532-4). In addition, detection by hybridisation has also been described (Olek et al., WO 99/28498).

Further publications dealing with the use of the bisulfite technique for methylation detection in individual genes are: Grigg G, Clark S. Sequencing 5-methylcytosine residues in genomic DNA. Bioessays. 1994 June; 16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March;6(3):387-95; Feil R, Charlton J, Bird A P, Walter J, Reik W. Methylation analysis on individual chromosomes: improved protocol for bisulphite genomic sequencing. Nucleic Acids Res. 1994 Feb. 25;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19;157(1-2):261-4; WO 97/46705, WO 95/15373 and WO 97/45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labelled probes are often used for the scanning of immobilised DNA arrays. The simple attachment of Cy3 and Cy5 dyes to the 5′-OH of the specific probe are particularly suitable for fluorescence labels. The detection of the fluorescence of the hybridised probes may be carried out, for example via a confocal microscope. Cy3 and Cy5 dyes, besides many others, are commercially available.

Matrix Assisted Laser Desorption Ionisation Mass Spectrometry (MALDI-TOF) is a very efficient development for the analysis of biomolecules (Karas M, Hillenkamp F. Laser desorption ionisation of proteins with molecular masses exceeding 10,000 daltons. Anal Chem. 1988 Oct. 15;60(20):2299-301). An analyte is embedded in a light-absorbing matrix. The matrix is evaporated by a short laser pulse thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionisation Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionisation process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. 1995 Apr. 25;23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

### DESCRIPTION

The invention provides a method and nucleic acids for the analysis of biological samples for features associated with the development of breast cell proliferative disorders. The invention is characterised in that the nucleic acid of at least one member of the group of genes according to Table 1 is/are contacted with a reagent or series of reagents capable of distinguishing between methylated and non methylated CpG dinucleotides within the genomic sequence of interest.

The present invention makes available a method for ascertaining genetic and/or epigenetic parameters of genomic DNA. The method is for use in the improved diagnosis, treatment and monitoring of breast cell proliferative disorders, for example by enabling the improved identification of and differentiation between subclasses of said disorder and the genetic predisposition to said disorders. The invention presents improvements over the state of the art in that it enables a highly specific classification of breast cell proliferative disorders, thereby allowing for improved and informed treatment of patients.

Furthermore, the method enables the analysis of cytosine methylations and single nucleotide polymorphisms.

The genes that form the basis of the present invention are preferably to be used to form a “gene panel”, i.e. a collection comprising the particular genetic sequences of the present invention and/or their respective informative methylation sites. The formation of gene panels allows for a quick and specific analysis of specific aspects of breast cancer. The gene panel(s) as described and employed in this invention can be used with surprisingly high efficiency for the diagnosis, treatment and monitoring of and the analysis of a predisposition to breast cell proliferative disorders.

In addition, the use of multiple CpG sites from a diverse array of genes allows for a relatively high degree of sensitivity and specificity in comparison to single gene diagnostic and detection tools.

The object of the invention is achieved by means of analysis of the methylation patterns of one or more sequences taken from the group comprising Seq. ID No. 1 through Seq. ID No. 73 and Seq. ID No. 366 according to Table 1. In a preferred embodiment said method is achieved by contacting said nucleic acid sequences in a biological sample obtained from a subject with at least one reagent or a series of reagents, wherein said reagent or series of reagents, distinguishes between methylated and non methylated CpG dinucleotides within the respective target nucleic acid(s), i.e. Seq. ID No. 1 through Seq. ID No. 73 and Seq. ID No. 366.

In a preferred embodiment, the method comprises the following steps:

In the first step of the method the genomic DNA sample must be isolated from sources such as cell lines, tissue or blood samples. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the next step of the method, this may be by any means standard in the state of the art, in particular, but not limited to, with restriction endonucleases.

In the second step of the method, the genomic DNA sample is treated in such a manner that cytosine bases which are unmethylated at the 5′-position are converted to uracil, thymine, or another base which is dissimilar to cytosine in terms of hybridisation behaviour. This will be understood as ‘pretreatment’ hereinafter.

The above described treatment of genomic DNA is preferably carried out with bisulfite (sulfite, disulfite) and subsequent alkaline hydrolysis which results in a conversion of non-methylated cytosine nucleobases to uracil or to another base which is dissimilar to cytosine in terms of base pairing behaviour. If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The converted DNA is then used for the detection of methylated cytosines.

Fragments of the pretreated DNA are amplified, using sets of primer oligonucleotides, and a preferably heat-stable, polymerase. Because of statistical and practical considerations, preferably more than ten different fragments having a length of 100-2000 base pairs are amplified. The amplification of several DNA segments can be carried out simultaneously in one and the same reaction vessel. Usually, the amplification is carried out by means of a polymerase chain reaction (PCR).

The design of such primers is obvious to one skilled in the art. These should include at least two oligonucleotides whose sequences are each reverse complementary or identical to an at least 18 base-pair long segment of the base sequences specified in the appendix (SEQ ID NO: 74 through SEQ ID NO: 365 and SEQ ID NO: 367 through 370). Said primer oligonucleotides are preferably characterised in that they do not contain any CpG dinucleotides. In a particularly preferred embodiment of the method, the sequence of said primer oligonucleotides are designed so as to selectively anneal to and amplify, only the breast cell specific DNA of interest, thereby minimising the amplification of background or non relevant DNA. In the context of the present invention, background DNA is taken to mean genomic DNA which does not have a relevant tissue specific methylation pattern, in this case, the relevant tissue being breast cells, both healthy and diseased.

According to the present invention, it is preferred that at least one primer oligonucleotide is bound to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminium, steel, iron, copper, nickel, silver, or gold, it being possible for other materials such as nitrocellulose or plastics to be used as well.

The fragments obtained by means of the amplification may carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualised by means of matrix assisted laser desorption/ionisation mass spectrometry (MALDI) or using electron spray mass spectrometry (ESI).

In the next step the nucleic acid amplificates are analysed in order to determine the methylation status of the genomic DNA prior to treatment.

The post treatment analysis of the nucleic acids may be carried out using alternative methods. Several methods for the methylation status specific analysis of the treated nucleic acids are described below, other alternative methods will be obvious to one skilled in the art.

Using several methods known in the art the analysis may be carried out during the amplification step of the method. In one such embodiment, the methylation status of preselected CpG positions within the nucleic acids comprising Seq. ID No. 1 through Seq. ID No. 73 may be detected by use of methylation specific primer oligonucleotides. This technique has been described in U.S. Pat. No. 6,265,171 to Herman. The use of methylation status specific primers for the amplification of bisulphite treated DNA allows the differentiation between methylated and unmethylated nucleic acids. MSP primers pairs contain at least one primer which hybridises to a bisulphite treated CpG dinucleotide. Therefore the sequence of said primers comprises at least one CG, TG or CA dinucleotide. MSP primers specific for non methylated DNA contain a ‘T’ at the 3′ position of the C position in the CpG. According to the present invention, it is therefore preferred that the base sequence of said primers is required to comprise a sequence having a length of at least 9 nucleotides which hybridises to a pretreated nucleic acid sequence according to Seq. ID No. 74 to Seq. ID No. 365 and sequences complementary thereto wherein the base sequence of said oligomers comprises at least one CG, TG or CA dinucleotide.

In one embodiment of the method the methylation status of the CpG positions may be determined by means of hybridisation analysis. In this embodiment of the method the amplificates obtained in the second step of the method are hybridised to an array or a set of oligonucleotides and/or PNA probes. In this context, the hybridisation takes place in the manner described as follows. The set of probes used during the hybridisation is preferably composed of at least 4 oligonucleotides or PNA-oligomers. In the process, the amplificates serve as probes which hybridise to oligonucleotides previously bonded to a solid phase. The non-hybridised fragments are subsequently removed. Said oligonucleotides contain at least one base sequence having a length of 10 nucleotides which is reverse complementary or identical to a segment of the base sequences specified in the appendix, the segment containing at least one CpG or TpG dinucleotide. In a further preferred embodiment the cytosine of the CpG dinucleotide, or in the case of TpG, the thiamine, is the 5^{th} to 9^{th} nucleotide from the 5′-end of the 10-mer. One oligonucleotide exists for each CpG or TpG dinucleotide.

The non-hybridised amplificates are then removed. In the final step of the method, the hybridised amplificates are detected. In this context, it is preferred that labels attached to the amplificates are identifiable at each position of the solid phase at which an oligonucleotide sequence is located.

In a further embodiment of the method the methylation status of the CpG positions may be ascertained by means of oligonucleotide probes that are hybridised to the treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996) employing a dual-labelled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which is designed to hybridise to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulphite treatment it is required that the probe be methylation specific, as described in U.S. Pat. No. 6,331,393, (hereby incorporated by reference) also known as the Methyl Light assay. Variations on the TaqMan™ detection methodology that are also suitable for use with the described invention include the use of dual probe technology (Lightcycler™ or fluorescent amplification primers (Sunrise™ technology). Both these techniques may be adapted in a manner suitable for use with bisulphite treated DNA, and moreover for methylation analysis within CpG dinucleotides.

A further suitable method for the use of probe oligonucleotides for the assessment of methylation by analysis of bisulphite treated nucleic acids is the use of blocker oligonucleotides. The use of such oligonucleotides has been described in BioTechniques 23(4), 1997, 714-720 D. Yu, M. Mukai, Q. Liu, C. Steinman. Blocking probe oligonucleotides are hybridised to the bisulphite treated nucleic acid concurrently with the PCR primers. PCR amplification of the nucleic acid is terminated at the 5′ position of the blocking probe, thereby amplification of a nucleic acid is suppressed wherein the complementary sequence to the blocking probe is present. The probes may be designed to hybridise to the bisulphite treated nucleic acid in a methylation status specific manner. For example, for detection of methylated nucleic acids within a population of unmethylated nucleic acids suppression of the amplification of nucleic acids which are unmethylated at the position in question would be carried out by the use of blocking probes comprising a ‘CG’ at the position in question, as opposed to a ‘CA’.

In a further preferred embodiment of the method the determination of the methylation status of the CpG positions is carried out by the use of template directed oligonucleotide extension, such as MS SNuPE as described by Gonzalgo and Jones (Nucleic Acids Res. 25:2529-2531).

In a further embodiment of the method the determination of the methylation status of the CpG positions is enabled by sequencing and subsequent sequence analysis of the amplificate generated in the second step of the method (Sanger F., et al., 1977 PNAS USA 74: 5463-5467).

A further embodiment of the invention is a method for the analysis of the methylation status of genomic DNA without the need for pretreatment. In the first and second steps of the method the genomic DNA sample must be obtained and isolated from tissue or cellular sources. Such sources may include cell lines, histological slides, body fluids, or tissue embedded in paraffin. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the treatment, this may be any means standard in the state of the art, in particular with restriction endonucleases. In the third step, the DNA is then digested with one or more methylation sensitive restriction enzymes. The digestion is carried out such that hydrolysis of the DNA at the restriction site is informative of the methylation status of a specific CpG dinucleotide.

In a preferred embodiment the restriction fragments are amplified. In a further preferred embodiment this is carried out using the polymerase chain reaction.

In the final step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis.

The aforementioned method is preferably used for ascertaining genetic and/or epigenetic parameters of genomic DNA.

In order to enable this method, the invention further provides the modified DNA of genes according to Table 1, as well as oligonucleotides and/or PNA-oligomers for detecting cytosine methylations within said genes. The present invention is based on the discovery that genetic and epigenetic parameters and, in particular, the cytosine methylation patterns of said genomic DNAs are particularly suitable for improved diagnosis, treatment and monitoring of breast cell proliferative disorders. Furthermore, the invention enables the differentiation between different subclasses of breast cell proliferative disorders or detection of a predisposition to breast cell proliferative disorders.

The nucleic acids according to the present invention can be used for the analysis of genetic and/or epigenetic parameters of genomic DNA.

This objective according to the present invention is achieved using a nucleic acid containing a sequence of at least 18 bases in length of the pretreated genomic DNA according to one of SEQ ID NO: 74 through SEQ ID NO: 365 and SEQ ID NO 367 through 370 and sequences complementary thereto.

The modified nucleic acids could heretofore not be connected with the ascertainment of disease relevant genetic and epigenetic parameters.

The object of the present invention is further achieved by an oligonucleotide or oligomer for the analysis of pretreated DNA, for detecting the genomic cytosine methylation state, said oligonucleotide containing at least one base sequence having a length of at least 10 nucleotides which hybridises to a pretreated genomic DNA according to SEQ ID NO: 74 through 365 and SEQ ID NO 367 through 370. The oligomer probes according to the present invention constitute important and effective tools which, for the first time, make it possible to ascertain specific genetic and epigenetic parameters during the analysis of biological samples for features associated with the development of breast cell proliferative disorders. Said oligonucleotides allow the improved diagnosis, treatment and monitoring of breast cell proliferative disorders and detection of the predisposition to said disorders. Furthermore, they allow the differentiation of different subclasses of breast carcinomas. The base sequence of the oligomers preferably contains at least one CpG or TpG dinucleotide. The probes may also exist in the form of a PNA (peptide nucleic acid) which has particularly preferred pairing properties. Particularly preferred are oligonucleotides according to the present invention in which the cytosine of the CpG dinucleotide is within the middle third of said oligonucleotide e.g. the 5^{th}-9^{th} nucleotide from the 5′-end of a 13-mer oligonucleotide; or in the case of PNA-oligomers, it is preferred for the cytosine of the CpG dinucleotide to be the 4^{th}-6^{th} nucleotide from the 5′-end of the 9-mer.

The oligomers according to the present invention are normally used in so called “sets” which contain at least one oligomer for each of the CpG dinucleotides within SEQ ID NO: 74 through SEQ ID NO: 365 and SEQ ID NO 367 through 370.

In the case of the sets of oligonucleotides according to the present invention, it is preferred that at least one oligonucleotide is bound to a solid phase. It is further preferred that all the oligonucleotides of one set are bound to a solid phase.

The present invention further relates to a set of at least 10 n (oligonucleotides and/or PNA-oligomers) used for detecting the cytosine methylation state of genomic DNA, by analysis of said sequence or treated versions of said sequence (according to SEQ ID NO: 1 through SEQ ID NO: 366 and sequences complementary thereto). These probes enable improved diagnosis, treatment and monitoring of breast cell proliferative disorders. In particular they enable the differentiation between different sub classes of breast cell proliferative disorders and the detection of a predisposition to said disorders.

The set of oligomers may also be used for detecting single nucleotide polymorphisms (SNPs) by analysis of said sequence or treated versions of said sequence according to one of SEQ ID NO: 1 through SEQ ID NO: 370.

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices are suitable alternatives.

Therefore, a further subject matter of the present invention is a method for manufacturing an array fixed to a carrier material for the improved diagnosis, treatment and monitoring of breast cell proliferative disorders, the differentiation between different subclasses of breast carcinomas and/or detection of the predisposition to breast cell proliferative disorders. In said method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the improved diagnosis, treatment and monitoring of breast cell proliferative disorders. Furthermore the DNA chip enables detection of the predisposition to breast cell proliferative disorders and the differentiation between different subclasses of breast carcinomas. The DNA chip contains at least one nucleic acid according to the present invention. DNA chips are known, for example, in U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond to or are complementary to a 18 base long segment of the base sequences specified in the appendix (SEQ ID NO: 74 through SEQ ID NO: 365 and SEQ ID NO 367 through 370), oligonucleotides and/or PNA-oligomers as well as instructions for carrying out and evaluating the described method.

In a further preferred embodiment said kit may further comprise standard reagents for performing a CpG position specific methylation analysis wherein said analysis comprises one or more of the following techniques: MS-SNuPE, MSP, Methyl light, Heavy Methyl, and nucleic acid sequencing. However, a kit along the lines of the present invention can also contain only part of the aforementioned components.

The oligomers according to the present invention or arrays thereof as well as a kit according to the present invention are intended to be used for the improved diagnosis, treatment and monitoring of breast cell proliferative disorders. Furthermore the use of said inventions extends to the differentiation between different subclasses of breast carcinomas and detection of the predisposition to breast cell proliferative disorders. According to the present invention, the method is preferably used for the analysis of important genetic and/or epigenetic parameters within genomic DNA, in particular for use in improved diagnosis, treatment and monitoring of breast cell proliferative disorders, detection of the predisposition to said disorders and the differentiation between subclasses of said disorders.

The methods according to the present invention are used, for example, for improved diagnosis, treatment and monitoring of breast cell proliferative disorders progression, detection of the predisposition to said disorders and the differentiation between subclasses of said disorders.

The present invention moreover relates to the diagnosis and/or prognosis of events which are disadvantageous or relevant to patients or individuals in which important genetic and/or epigenetic parameters within genomic DNA, said parameters obtained by means of the present invention may be compared to another set of genetic and/or epigenetic parameters, the differences serving as the basis for the diagnosis and/or prognosis of events which are disadvantageous or relevant to patients or individuals.

The genes and/or the nucleic acids that form the basis of the present invention can be used to form a “gene panel”, i.e. a collection comprising the particular genetic sequences of the present invention and/or their respective informative methylation sites. The formation of gene panels allow for a quick and specific analysis of the disorders they are related with. The gene panel(s) as described and employed in this invention can be used with surprisingly high efficiency for the diagnosis, treatment and monitoring of and the analysis of a predisposition to the disorders described herein, based on the analysis of the methylation status of said panels.

The use of multiple and selective CpG sites from a diverse array of genes regulating breast cell proliferative disorders, in addition allows for a surprisingly high degree of sensitivity and specificity in comparison to single gene diagnostic and detection tools. Furthermore, as compared to many the panel as described herein may be adapted for use in the analysis of multiple diseases all affected by regulating breast cell proliferative disorders.

In the context of the present invention the term “hybridisation” is to be understood as a bond of an oligonucleotide to a completely complementary sequence along the lines of the Watson-Crick base pairings in the sample DNA, forming a duplex structure.

In the context of the present invention, “genetic parameters” are mutations and polymorphisms of genomic DNA and sequences further required for their regulation. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations and further modifications of DNA bases of genomic DNA and sequences further required for their regulation. Further epigenetic parameters include, for example, the acetylation of histones which, cannot be directly analysed using the described method but which, in turn, correlates with the DNA methylation.

In the following, the present invention will be explained in greater detail on the basis of the sequences, the tables, and the examples without being limited thereto. SEQ ID NO: 1 through SEQ ID NO: 73 and SEQ ID NO: 366 represent 5′ and/or regulatory regions and/or CpG rich regions of the genes according to Table 1. These sequences are derived from Genbank and will be taken to include all minor variations of the sequence material which are currently unforeseen, for example, but not limited to, minor deletions and SNPs.

SEQ ID NO: 74 through SEQ ID NO: 365 and SEQ ID NO 367 through 370 exhibit the pretreated sequence of DNA derived from the genes according to Table 1. These sequences will be taken to include all minor variations of the sequence material which are currently unforeseen, for example, but not limited to, minor deletions and SNPs.

SEQ ID NO: 371 through SEQ ID NO: 396 exhibit the sequences of primers and other oligonucleotides used in the analysis of a selected panel of the genes of Table 1, as described in the embodiments of the method according to examples 1 to 4.

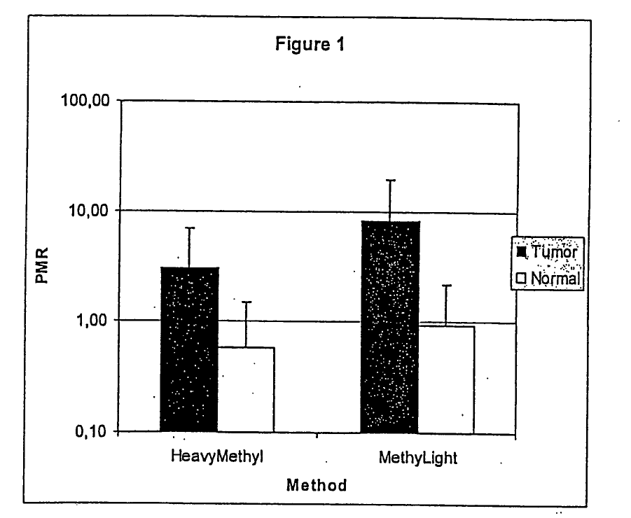

FIG. 1 shows the analysis of bisulphite-treated DNA using the MethylLight and Heavy Methyl assays, performed according to Examples 2 and 3 respectively. Results of the HeavyMethyl assay are shown on the left hand bar chart and results of the MethyLight assay are shown on the right hand bar chart. The Y-axis shows the percentage of methylation at the CpG positions covered by the probes. The dark black bar (“A” in the legend) corresponds to tumour samples, whereas the white bar (“B”) corresponds to normal control tissue. Significantly, the tumour samples are substantially hypermethylated relative to normal control tissue.

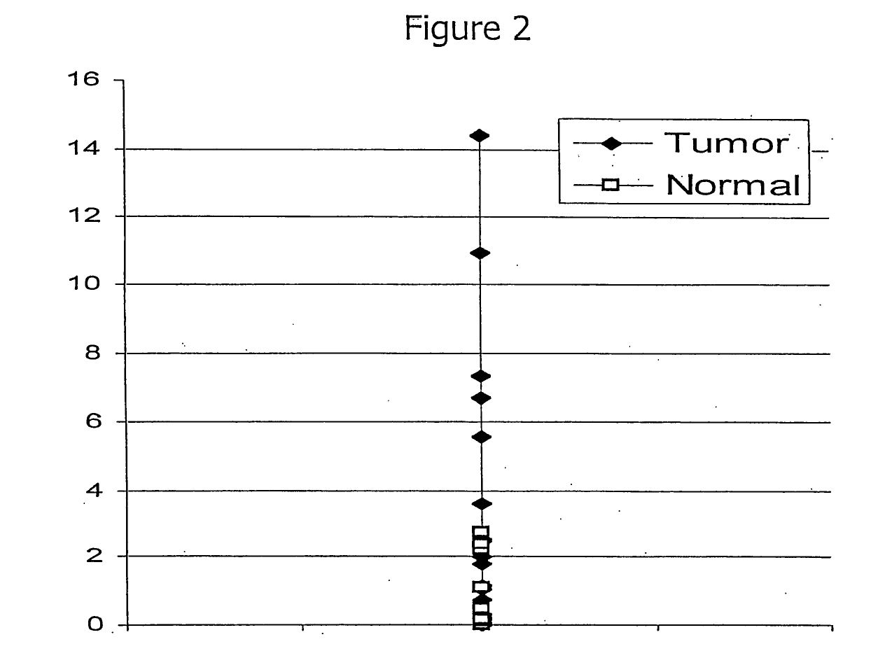

FIG. 2 shows the level of methylation in breast tumour and healthy tissues as assessed according to Example 3 by means of the Heavy Methyl assay. The Y-axis shows the degree of methylation within the region of the Calcitonin gene investigated. Tumour samples are represented by black diamonds, and normal breast tissue samples by white squares. As can be seen from the results, a significantly higher degree of methylation (hypermethylation) was observed in tumour samples relative to normal tissue samples.

TABLE 1
Description of genes comprising panel.

| | | | | Seq ID | Seq. ID No. |
|---|---|---|---|---|---|
| Accession | | | Seq. ID No. | No. Treated | Treated |
| No. | Gene name | Description | (Genomic) | (methylated) | (unmethylated) |
| NM_001846 | COL4A2 | collagen, type IV, | 1 | 74, 75 | 82, 83 |
| | | alpha 2 | | | |
| NM_004663 | GTPase | rab11a GTPase | 2 | 76, 77 | 84, 85 |
| NM_001218 | CA XII | carbonic anhydrase | 3 | 78, 79 | 86, 87 |
| | | XII precursor | | | |
| AF073519 | SERF1A | small EDRK-rich | 4 | 80, 81 | 88, 89 |
| | | factor 1A (telomeric) | | | |
| NM_006526 | ZNF217 | Zinc Finger Protein | 5 | 90, 91 | 96, 97 |
| | | 217 | | | |
| AJ293618 | JCL-1 | Human | 6 | 92, 93 | 98, 99 |
| | | hepatocellular | | | |
| | | carcinoma associated | | | |
| | | protein | | | |
| NM_007194 | CHK2 | SERINE/THREONINE- | 7 | 94, 95 | 100, 101 |
| | | PROTEIN | | | |
| | | KINASE CHK2 (EC | | | |
| | | 2.7.1.—)(CDS1). | | | |
| D14034 | zinc-alpha2- | | 8 | 102, 103 | 114, 115 |
| | glycoprotein | | | | |
| X03473 | Histone H1 | Human gene for | 9 | 104, 105 | 116, 117 |
| | | histone H1(0) | | | |
| NM_000127 | EXT1 | exostoses (multiple) 1 | 10 | 106, 107 | 118, 119 |
| NM_000436 | OXCT | 3-oxoacid CoA | 11 | 108, 109 | 120, 121 |
| | | transferase | | | |
| NM_000599 | IGFBP5 | insulin-like growth | 12 | 110, 111 | 122, 123 |
| | | factor binding | | | |
| | | protein 5 | | | |
| NM_000849 | GSTM3 | glutathione S- | 13 | 112, 113 | 124, 125 |
| | | transferase M3 | | | |
| | | (brain) | | | |
| NM_001282 | AP2B1 | adaptor-related | 14 | 126, 127 | 138, 139 |
| | | protein complex 2, | | | |
| | | beta 1 subunit | | | |
| NM_001809 | CENPA | centromere protein | 15 | 128, 129 | 140, 141 |
| | | A(17 kD) | | | |
| NM_002019 | FLT1 | fms-related tyrosine | 16 | 130, 131 | 142, 143 |
| | | kinase 1 (vascular | | | |
| | | endothelial growth | | | |
| | | factor/vascular | | | |
| | | permeability factor | | | |
| | | receptor) | | | |
| NM_002073 | GNAZ | guanine nucleotide | 17 | 132, 133 | 144, 145 |
| | | binding protein (G | | | |
| | | protein), alpha z | | | |
| | | polypeptide | | | |
| NM_002916 | RFC4 | replication factor C | 18 | 134, 135 | 146, 147 |
| | | (activator 1) 4 | | | |
| | | (37 kD) | | | |
| NM_003239 | TGFB3 | transforming growth | 19 | 136, 137 | 148, 149 |
| | | factor, beta 3 | | | |
| NM_003607 | PK428 | Ser-Thr protein | 20 | 150, 151 | 162, 163 |
| | | kinase related to the | | | |
| | | myotonic dystrophy | | | |
| | | protein kinase | | | |
| NM_003748 | ALDH4 | aldehyde | 21 | 152, 153 | 164, 165 |
| | | dehydrogenase 4 | | | |
| | | (glutamate gamma- | | | |
| | | semialdehyde | | | |
| | | dehydrogenase | | | |
| NM_003875 | GMPS | guanine | 22 | 154, 155 | 166, 167 |
| | | monphosphate | | | |
| | | synthetase | | | |
| NM_003882 | WISP1 | WNT1 inducible | 23 | 156, 157 | 168, 169 |
| | | signaling pathway | | | |
| | | protein 1 | | | |
| NM_003981 | PRC1 | protein regulator of | 24 | 158, 159 | 170, 171 |
| | | cytokinesis 1 | | | |
| NM_004702 | CCNE2 | G1/S-Specific | 25 | 160, 161 | 172, 173 |
| | | Cyclin E2. | | | |
| NM_004994 | MMP9 | matrix | 26 | 174, 175 | 184, 185 |
| | | metalloproteinase 9 | | | |
| | | (gelatinase B, 92 kD | | | |
| | | gelatinase, 92 kD | | | |
| | | type IV collagenase) | | | |
| NM_005915 | MCM6 | minichromosome | 27 | 176, 177 | 186, 187 |
| | | maintenance | | | |
| | | deficient (mis5, S. pombe)6 | | | |
| NM_006101 | HEC | highly expressed in | 28 | 178, 179 | 188, 189 |
| | | cancer, rich in | | | |
| | | leucine heptad | | | |
| | | repeats | | | |
| NM_006117 | PECI | peroxisomal D3, D2-. | 29 | 180, 181 | 190, 191 |
| | | enoyl-CoA | | | |
| | | isomerase | | | |
| NM_006681 | NMU | neuromedin U | 30 | 182, 183 | 192, 193 |
| NM_006931 | SLC2A3 | solute carrier family | 31 | 194, 195 | 204, 205 |
| | | 2 (facilitated glucose | | | |
| | | transporter), member 3 | | | |
| NM_007036 | ESM1 | endothelial cell- | 32 | 196, 197 | 206, 207 |
| | | specific molecule 1 | | | |
| NM_007203 | AKAP2 | A kinase (PRKA) | 33 | 198, 199 | 208, 209 |
| | | anchor protein 2 | | | |
| NM_020188 | DC13 | DC13 protein | 34 | 200, 201 | 210, 211 |
| NM_015984 | UCH37 | ubiquitin C-terminal | 35 | 202, 203 | 212, 213 |
| | | hydrolase UCH37 | | | |
| NM_014321 | ORC6L | origin recognition | 36 | 214, 215 | 218, 219 |
| | | complex, subunit 6 | | | |
| | | (yeast homolog)-like | | | |
| NM_016448 | L2DTL | L2DTL protein | 37 | 216, 217 | 220, 221 |
| NM_000038 | APC | adenomatosis | 38 | 222, 223 | 240, 241 |
| | | polyposis coli | | | |
| NM_006768 | BRCA1 | breast cancer 1, early | 39 | 224, 225 | 242, 243 |
| | | onset | | | |
| NM_001759 | CCND2 | cyclin D2 | 40 | 226, 227 | 244, 245 |
| NM_004360 | CDH1 | cadherin 1, type 1, | 41 | 228, 229 | 246, 247 |
| | | E-cadherin | | | |
| NM_001257 | CDH13 | H-cadherin | 42 | 230, 231 | 248, 249 |
| NM_058195, | CDKN2A | cyclin-dependent | 43 | 232, 233 | 250, 251 |
| NM_058196, | | kinase inhibitor 2A | | | |
| NM_000077 | | | | | |
| NM_000125 | ESR1 | estrogen receptor 1 | 44 | 234, 235 | 252, 253 |
| NM_004102 | FABP3 | fatty acid binding | 45 | 236, 237 | 254, 255 |
| | | protein 3 | | | |
| NM_002012 | FHIT | fragile histidine triad | 46 | 238, 239 | 256, 257 |
| | | gene | | | |
| NM_004004 | GJB2 | gap junction protein, | 47 | 258, 259 | 276, 277 |
| | | beta 2 | | | |
| NM_000852 | GSTP1 | Glutathione S- | 48 | 260, 261 | 278, 279 |
| | | transferase pi | | | |
| NM_006497 | HIC-1 | hypermethylated in | 49 | 262, 263 | 280, 281 |
| | | cancer 1 | | | |
| NM_002478 | MYOD1 | myogenic factor 3 | 50 | 264, 265 | 282, 283 |
| NM_007182 | RASSF1 | Ras association | 51 | 266, 267 | 284, 285 |
| | | (RalGDS/AF-6) | | | |
| | | domain family 1 | | | |
| NM_133631, | ROBO1 | roundabout, axon | 52 | 268, 269 | 286, 287 |
| NM_002941 | | guidance receptor, | | | |
| | | homolog 1 | | | |
| NM_006142 | SFN | stratifin | 53 | 270, 271 | 288, 289 |
| NM_013258 | TMS1 | target of | 54 | 272, 273 | 290, 291 |
| | | methylation-induced | | | |
| | | silencing gene | | | |
| NM_004906 | WT1 | Wilms tumor 1 | 55 | 274, 275 | 292, 293 |
| NM_016359 | LOC51203 | clone HQ0310 | 56 | 294, 295 | 310, 311 |
| | | PRO0310p1 | | | |
| NM_014791 | KIAA0175 | KIAA0175 gene | 57 and 58 | 296-299 | 312-315 |
| | | product | | | |
| NM_016577 | RAB6B | RAB6B, member | 59 | 300, 301 | 316, 317 |
| | | RAS oncogene | | | |
| | | family | | | |
| NM_000788 | DCK | deoxycytidine kinase | 60 | 302, 303 | 318, 319 |
| NM_014889 | MP1 | metalloprotease 1 | 61 | 304, 305 | 320, 321 |
| | | (pitrilysin family) | | | |
| NM_000599 | IGFBP5 | insulin-like growth | 62 | 306, 307 | 322, 323 |
| | | factor binding | | | |
| | | protein 5 | | | |
| NM_020386 | LOC57110 | H-REV107 protein- | 63 | 308, 309 | 324, 325 |
| | | related protein | | | |
| NM_018401 | HSA250839 | gene for | 64 | 326, 327 | 340, 341 |
| | | serine/threonine | | | |
| | | protein kinase | | | |
| BC001653 | MG2771 | hypothetical protein | 65 | 328, 329 | 342, 343 |
| | | MG2771 | | | |
| X51754 | IG | Lambda- | 66 | 330, 331 | 344, 345 |
| | LAMBDA | immunoglobulin | | | |
| | CHAIN C | light chain. | | | |
| | REGIONS | | | | |
| U82987 | BBC3 | Bcl-2 binding | 67 | 332, 333 | 346, 347 |
| | | component 3 | | | |
| X60111 | HMRP-1 | H. sapiens MRP-1 | 68 | 334, 335 | 348, 349 |
| AB021868 | STAT3 | signal transducer and | 69 | 336, 337 | 350, 351 |
| | | activator of | | | |
| | | transcription 3 | | | |
| X55543 | TREB | tax-responsive | 70 | 338, 339 | 352, 353 |
| | | element-binding | | | |
| | | protein | | | |
| NM_005978 | s100a2 | calcium-binding | 71 | 354, 355 | 360, 361 |
| | | protein S100A2 | | | |
| NM_002658 | UPA | Urokinase | 72 | 356, 357 | 362, 363 |
| NM_000926 | PGR | progesterone | 73 | 358, 359 | 364, 365 |
| | | receptor | | | |
| X15943 | Calcitonin | Calcitonin | 366 | 367, 368 | 370, 369 |

### EXAMPLES

In order to establish the suitability of genes for inclusion within the panel each gene was investigated by means of both relatively unspecific and highly sensitive methods. Methylation within the Calcitonin gene was analysed using three different methods, namely restriction enzyme, MethyLight and combined HeavyMethyl MethyLight assays.

### Example 1

### Restriction Enzyme Analysis

The differential methylation was initially observed by means of methylation sensitive restriction enzyme analysis. A fragment of the upstream region of the calcitonin gene (SEQ ID NO:366) was amplified by PCR using the primers CCTTAGTCCCTACCTCTGCT (SEQ ID NO:371) and CTCATTTACACACACCCAAAC (SEQ ID NO:372). The resultant amplificate, 378 bp in length, contained an informative CpG at position 165. The amplificate DNA was digested with the methylation sensitive restriction endonuclease Nar I; recognition motif GGCGCC. Hydrolysis by said endonuclease is blocked by methylation of the CpG at position 165 of the amplificate. The digest was used as a control.

Genomic DNA was isolated from the breast tissue and breast tumour samples using the DNA wizzard™ DNA isolation kit (Promega). Each sample was digested using Nar I according to manufacturer's recommendations (New England Biolabs).

About 10 ng of each genomic digest was then amplified using PCR primers CCTTAGTCCCTACCTCTGCT (SEQ ID NO: 371) and CTCATTTACACACACCCAAAC (SEQ ID NO: 372). The PCR reactions were performed using a thermocycler (Eppendorf GmbH) using 10 ng of DNA, 6 pmole of each primer, 200 μM of each DNTP, 1.5 mM MgCl₂ and 1 U of Hotstart™Taq (Qiagen AG). The other conditions were as recommended by the Taq polymerase manufacturer.

Using the above mentioned primers, gene fragments were amplified by PCR performing a first denaturation step for 14 min at 96° C., followed by 30-45 cycles (step 2: 60 sec at 96° C., step 3: 45 sec at 52° C., step 4: 75 sec at 72° C.) and a subsequent final elongation of 10 min at 72° C. The presence of PCR products was analysed by agarose gel electrophoresis.

PCR products were detectable, with Nar 1-hydrolysed DNA isolated wherein the tissue in question contained upmethylated DNA, when step 2 to step 4 of the cycle program were repeated 34, 37, 39, 42 and 45 fold. In contrast, PCR products were only detectable with Nar I-hydrolyzed DNA isolated from downmethylated tissue when steps 2 to step 4 of the cycle program were repeated 42- and 45-fold. Further investigation of the Calcitonin gene was then carried out by means of highly sensitive assays as described in Example 2 and 3.

### Example 2

### Analysis of Methylation within Breast Cancer Using a MethyLight Assay

DNA was extracted from 21 breast carcinoma samples and 17 normal breast tissues using a Qiagen extraction kit. The DNA from each sample was treated using a bisulfite solution (hydrogen sulfite, disulfite) according to the agarose-bead method (Olek et al 1996). The treatment is such that all non methylated cytosines within the sample are converted to thymidine. Conversely, 5-methylated cytosines within the sample remain unmodified.

The methylation status was determined with a MethyLight assay designed for the CpG island of interest and a control fragment from the beta actin gene (Eads et al., 2001). The CpG island assay covers CpG sites in both the primers and the taqman style probe, while the control gene does not. The control gene is used as a measure of total DNA concentration, and the CpG island assay (methylation assay) determines the methylation levels at that site.

Methods. The Calcitonin gene CpG island assay was performed using the following primers and probes:

| | |
|---|---|
| Primer: | |
| AGGTTATCGTCGTGCGAGTGT; | (SEQ ID NO:373) |
| Primer: | |
| TCACTCAAACGTATCCCAAACCTA; | (SEQ ID NO:374) |
| and | |
| Probe: | |
| CGAATCTCTCGAACGATCGCATCCA. | (SEQ ID NO:375) |

The corresponding control assay was performed using the following primers and probes

| | |
|---|---|
| Primer: | |
| TGGTGATGGAGGAGGTTTAGTAAGT; | (SEQ ID NO:376) |
| Primer: | |
| AACCAATAAAACCTACTCCTCCCTTAA; | (SEQ ID NO:377) |
| and | |
| Probe: | |
| ACCACCACCCAACACACAATAACAAACACA. | (SEQ ID NO:378) |

The reactions were run in triplicate on each DNA sample with the following assay conditions:

Reaction solution: (900 nM primers; 300 nM probe; 3.5 mM Magnesium Chloride; 1 unit of taq polymerase; 200 mM dNTPs; 7 ml of DNA, in a final reaction volume of 20 ml);

Cycling conditions: (95° C. for 10 minutes; 95° C. for 15 seconds; 67° C. for 1 minute (3 cycles)); (95° C. for 15 seconds, 64° C. for 1 minute (3 cycles)); (95° C. for 15 seconds, 62° C. for 1 minute (3 cycles)); and (95° C. for 15 seconds, 60° C. for 1 minute (40 cycles)). The data was analysed using a PMR calculation previously described in the literature (Eads et al 2001).

Results. The mean PMR for normal samples was 0.94, with a standard deviation of 1.28. The mean PMR for tumour samples was 8.38, with a standard deviation of 11.18. The overall difference in methylation levels between tumour and normal samples is significant in a t-test (p=0.0065).

### Experiment 3

### Single Gene Analysis

The same samples were analysed using the HeavyMethyl MethyLight (or HM MethyLight) assay, also referred to as the HeavyMethyl assay. The methylation status was determined with a HM MethyLight assay designed for the CpG island of interest and a control gene assay. The CpG island assay covers CpG sites in both the blockers and the taqman style probe, while the control gene does not.

Methods. The CpG island assay (methylation assay) was performed using the following primers and probes:

| | |
|---|---|
| Primer: | |
| GGATGTGAGAGTTGTTGAGGTTA; | (SEQ ID NO: 379) |
| Primer: | |
| ACACACCCAAACCCATTACTATCT; | (SEQ ID NO: 380) |
| Probe: | |
| ACCTCCGAATCTCTCGAACGATCGC; | (SEQ ID NO: 381) |
| and | |
| Blocker: | |
| TGTTGAGGTTATGTGTAATTGGGTGTGA. | (SEQ ID NO: 382) |

The reactions were each run in triplicate on each DNA sample with the following assay conditions:

Reaction solution: (300 nM primers; 450 nM probe; 3.5 mM magnesium chloride; 2 units of taq polymerase; 400 mM dNTPs; and 7 ml of DNA, in a final reaction volume of 20 ml);

Cycling conditions: (95° C. for 10 minutes); (95° C. for 15 seconds, 67° C. for 1 minute (3 cycles)); (95° C. for 15 seconds, 64° C. for 1 minute (3 cycles); (95° C. for 15 seconds, 62° C. for 1 minute (3 cycles)); and (95° C. for 15 seconds, 60° C. for 1 minute (40 cycles)).

Results. The mean PMR for normal samples was 0.58, with a standard deviation of 0.94. The mean PMR for tumour samples was 3.01, with a standard deviation of 3.91. The overall difference in methylation levels between tumor and normal samples is significant in a t-test (p=0.0012).

Taking into account the significance of the analyses it was decided to combine the identified CpG island with other informative CpG rich regions in the form of a gene panel for use as a diagnostic assay. This would increase the level of specificity and sensitivity as opposed to use of the identified CpG rich region as a single gene marker type diagnostic assay.

### Example 4

### Multiple Gene ‘Panel’ Analysis

The informative region as disclosed in SEQ ID No. 366 was included in a panel of genes for the assessment of breast tumours samples. Accordingly, an assay was devised suitable for the medium throughput analysis of mutliple CpG positions in multiple samples, the chosen format being microarray analysis.

All samples were treated using the bisulphite technique disclosed in ‘Example 1’. Following bisulphite treatment selected CpG rich regions from the genes according to Table 2 were amplified by means of multiplex polymerase chain reaction, amplifying 8 fragments per reaction with Cy5 fluorescently labelled primers according to Table 2. The following conditions were used:

10 ng bisulfite treated DNA

3.5 mM MgCl₂

400 μM dNTPs

2 pmol each primer

1 U Hot Start Taq (Qiagen)

Forty cycles were carried out as follows. Denaturation at 95° C. for 15 min, followed by annealing at 55° C. for 45 sec., primer elongation at 65° C. for 2 min. A final elongation at 65° C. was carried out for 10 min.

All PCR products from each individual sample were then hybridised to glass slides carrying a pair of immobilised oligonucleotides for each CpG position under analysis. Each of these detection oligonucleotides was designed to hybridise to the bisulphite converted sequence around one CpG site which was either originally unmethylated (TG) or methylated (CG). Hybridisation conditions were selected to allow the detection of the single nucleotide differences between the TG and CG variants.

5 μl volume of each multiplex PCR product was diluted in 10×Ssarc buffer (10×Ssarc:230 ml 20×SSC, 180 ml sodium lauroyl sarcosinate solution 20%, dilute to 1000 ml with dH₂O). The reaction mixture was then hybridised to the detection oligonucleotides as follows. Denaturation at 95° C., cooling down to 10° C., hybridisation at 42° C. overnight followed by washing with 10×Ssarc and dH₂O at 42° C.

Fluorescent signals from each hybridised oligonucleotide were detected using genepix scanner and software. Ratios for the two signals (from the CG oligonucleotide and the TG oligonucleotide used to analyse each CpG position) were calculated based on comparison of intensity of the fluorescent signals.

The information is then sorted into a ranked matrix according to CpG methylation differences between the two classes of tissues, using an algorithm. In order to accurately discriminate between the two classes of tissues, we trained a learning algorithm (support vector machine, SVM). The SVM was discussed by F. Model, P. Adorjan, A. Olek, C. Piepenbrock, Feature selection for DNA methylation based cancer classification. Bioinformatics. 2001 June;17 Suppl 1:S157-64. The algorithm constructs an optimal discriminant between two classes of given training samples. In this case each sample is described by the methylation patterns (CG/TG ratios) at the investigated CpG sites. The SVM was trained on a subset of samples of each class, which were presented with the diagnosis attached. Independent test samples, which were not shown to the SVM before were then presented to evaluate, to establish if the diagnosis could be predicted correctly based on the predictor created in the training round. This procedure was repeated several times using different partitions of the samples, a method called cross-validation. Please note that all rounds are performed without using any knowledge obtained in the previous runs. The number of correct classifications was averaged over all runs, which gives a good estimate of our test accuracy (percent of correct classified samples over all rounds).

TABLE 2
Genes and Primers according to Example 4

| | |
|---|---|
| Gene | Primers |

| | |
|---|---|
| CDKN2A | TTGAAAATTAAGGGTTGAGG |
| SEQ ID NO 43 | (SEQ ID NO: 383) |
| | CACCCTCTAATAACCAACCA |
| | (SEQ ID NO: 384) |
| CDKN2A | GGGGTTGGTTGGTTATTAGA |
| SEQ ID NO 43 | (SEQ ID NO: 385) |
| | AACCCTCTACCCACCTAAAT |
| | (SEQ ID NO: 386) |
| RASSF1 | ACCTCTCTACAAATTACAAATTCA |
| SEQ ID NO 51 | (SEQ ID NO: 387) |
| | AGTTTGGGTTAGTTTGGGTT |
| | (SEQ ID NO: 388) |
| MYOD1 | ATTAGGGGTATAGAGGAGTATTGA |
| SEQ ID NO 50 | (SEQ ID NO: 389) |
| | CTTACAAACCCACAATAAACAA |
| | (SEQ ID NO: 390) |
| WT1 | AAAGGGAAATTAAGTGTTGT |
| SEQ ID NO 55 | (SEQ ID NO: 391) |
| | TAACTACCCTCAACTTCCC |
| | (SEQ ID NO: 392) |
| BRCA1 | TGGATGGGAATTGTAGTTTT |
| SEQ ID NO 39 | (SEQ ID NO: 393) |
| | TTAACCACCCAATCTACCC |
| | (SEQ ID NO: 394) |
| CCND2 | TTTTGGTATGTAGGTTGGATG |
| SEQ ID NO 40 | (SEQ ID NO: 395) |
| | CCTAACCTCCTTCCTTTAACT |
| | (SEQ ID NO: 396) |
| Calcitonin | AGGTTATCGTCGTGCGAGTGT |
| SEQ ID NO: 366 | (SEQ ID NO: 373) |
| | TCACTCAAACGTATCCCAAACCTA |
| | (SEQ ID NO: 374) |

1. A method for the analysis of breast cell proliferative disorders, comprising determining the genomic methylation status of at least one CpG dinucleotide of at least one sequence selected from the sequence group consisting of SEQ ID NOS:1-73, SEQ ID NO:366, contiguous portions thereof, and sequences complementary thereto.

2. The method of claim 1, wherein determining the genomic methylation status of the at least one CpG dinucleotide, comprises: obtaining a biological sample comprising genomic DNA from a subject; and contacting the genomic DNA with at least one reagents or a series of reagent, which distinguishes between methylated and non-methylated CpG dinucleotides.

3. A nucleic acid molecule consisting essentially of a sequence at least 18 contiguous bases in length of a sequence selected from the sequence group consisting of SEQ ID NOS:74-365, and SEQ ID NOS:367-370, and sequences complementary thereto.

4. An oligomer, consisting essentially of a sequence of at least 10 contiguous nucleotides in length that hybridises to or is identical to a sequence selected from the group consisting of SEQ ID NOS:1-370.

5. The oligomer of claim 4, wherein the contiguous base sequence includes at least one CpG dinucleotide.

6. The oligomer of claim 5, wherein the cytosine of the CpG dinucleotide is located in about the middle third of the oligomer.

7. A set of oligomers, comprising at least two oligomers according to any of claims 4 to 6.

8. The set of oligomers of claim 7, comprising oligomers for detecting the methylation state of all CpG dinucleotides within sequences of the sequence group consisting of SEQ ID NOS: 1-73 SEQ ID NO: 366, contiguous portions thereof, and sequences complementary thereto.

9. The set of oligomers of claim 7, wherein the set is suitable for use as primer oligonucleotides for the amplification of a sequence selected from the sequence group consisting of SEQ ID NOS: 1-370, contiguous portions thereof, and sequences complementary thereto.

10. The set of oligomers according to any one of claims 7 through 9, wherein at least one oligomer is bound to a solid phase.

11. A method for determining methylation state or for detecting single nucleotide polymorphisms, comprising using a set of oligonucleotides comprising at least three oligomers according to any of claims 4 through 10 in an assay suitable for at least one of detecting cytosine methylation state single nucleotide polymorphisms (SNPs), within a sequence selected from the group consisting of SEQ ID NOS: 1-370, contiguous portions thereof, and sequences complementary thereto.

12. A method for manufacturing an arrangement of different oligomers (array) fixed to a carrier material and suitable for analysing breast cell proliferative disorders associated with the methylation state of of at least one CpG dinucleotide of a sequence selected from the group consisting of SEQ ID NOS:1-73 and SEQ ID NO:366, contiguous portions thereof, and sequences complementary thereto, comprising coupling at least one nucleic acid or oligomer according to any of the claims 3 through 10 to a solid phase.

13. An arrangement of different oligomers (array) obtainable according to claim 12.

14. The arrangement of claim 13 wherein the oligomers are at least one of oligonucleotides and PNA-oligomer sequences, wherein the carrier material is a planar solid phase, and wherein the oligomers are arranged thereon in the form of a rectangular or hexagonal lattice.

15. The arrangement of claim 13, wherein the carrier material comprises a material selected from the group consisting of silicon, glass, polystyrene, aluminium, steel, iron, copper, nickel, silver, gold, and combinations thereof.

16. An oligomer array suitable for analysing breast cell proliferative disorders associated with the methylation state of at least one CpG dinucleotide of a sequence selected from the group consisting of SEQ ID NOS:1-73, SEQ ID NO:366, contiguous portions thereof, and sequences complementary thereto, the array comprising at least one nucleic acid or oligomer according to any one of claims 3 through 10.

17. A method for the analysis of breast cell proliferative disorders, comprising:
a) obtaining a biological sample comprising genomic DNA:
b) contacting the genomic DNA, or a portion thereof with an agent or combination of agents suitable to convert cytosine bases that are unmethylated at the 5-position to uracil or to another base which is dissimilar to cytosine in terms of hybridisation behaviour, to provide a pretreated DNA;
c) amplifying, using at least one set of primer oligonucleotides and a polymerase, at least one pretreated DNA sequence, or a portion thereof, selected from the sequence group consisting of SEQ ID NOS:74-365, SEB ID NOS:367-370, contiguous portions thereof, and sequences complementary thereto;
d) determining, based on the amplification, or on analysis of the nucleic acid amplificate, the methylation status of one or more genomic CpG dinucleotides, whereby analysis of breast cell proliferative disorders is, at least in part, afforded.

18. The method of claim 17, wherein determining in d) comprises hybridisation of at least one nucleic acid or oligomer according to any one of claims 3 through 10.

19. The method of claim 17, wherein determining in d) comprises hybridisation of at least one oligonucleotide according to any one of claims 3 through 10, and extension of the at least one hybridised oligonucleotide by at least one nucleotide base.

20. The method of claim 17, wherein determining in d) comprises sequencing.

21. The method of claim 17, wherein amplifying in c) comprises using methylation-specific primers.

22. The method of claim 17, wherein determining in d) comprises use of a combination of at least two of the methods described in any one of claims 18 through 21.

23. The method of claim 17, wherein contacting in c) comprises contacting with at least one agent selected from the group consisting of bisulfite, hydrogen sulfite or disulfite.

24. A method for the analysis of breast cell proliferative disorders, comprising:
a) obtaining, from a subject, a biological sample containing genomic DNA;
b) isolating the genomic DNA;
c) digesting the isolated genomic DNA, or a portion thereof comprising at least one sequence selected from the sequence group consisting of SEQ ID NOS1-73, SEQ ID NO:366, contiguous portions thereof, and sequences complementary thereto, with one or more methylation-sensitive methylation sensitive restriction enzymes; and
d) detecting of the DNA fragments generated in c), whereby analysis of breast cell proliferative disorders is, at least in part, afforded.

25. The method of according to claim 24, further comprising, prior to d), amplifying the DNA digest.

26. The method of any one of claims 17 and 25, wherein more than ten different fragments having a length of about 100 to about 200 base pairs are amplified.

27. The method of any one of claims 17 and 26, wherein the amplification of several DNA segments is carried out in one reaction vessel.

28. The method of any one of claims 17 and 25, wherein amplifying is by means of a heat-resistant DNA polymerase.

29. The method of any one of claims 17 and 25, wherein amplifying is by means of a polymerase chain reaction (PCR).

30. The method of any one of claims 17 and 25, wherein the amplificates carry detectable labels.

31. The method of claim 30, wherein said labels are selected from the group consisting of fluorescence labels, radionuclides, detachable molecule fragments having a typical mass which can be detected in a mass spectrometer and combinations thereof.

32. The method of claim 17, wherein amplificates or fragments of the amplificates are detected in the mass spectrometer.

33. The method of any one of claims 31 and 32, wherein produced fragments have a single positive or negative net charge for better detectability in the mass spectrometer.

34. The method of claim 30, wherein detection is carried out and visualised by means of at least one of matrix assisted laser desorption/ionisation mass spectrometry (MALDI), and using electron spray mass spectrometry (ESI).

35. The method of any one of the claims 17 and 24, wherein the genomic DNA is obtained from cells or cellular components which contain DNA, sources of DNA comprising, for example, cell lines, histological slides, biopsies, tissue embedded in paraffin and all possible combinations thereof.

36. A kit reagent having at least one of bisulfite, disulfite, and hydrogen sulfite, as well as at least one of oligonucleotides, and PNA-oligomers according to any one of the claims 4 through 10.

37. The kit of claim 36, further comprising standard reagents for performing a methylation assay selected from the group consisting of MS-SNuPE, MSP, MethyLight, HeavyMethyl, nucleic acid sequencing, and combinations thereof.

38. (canceled)

39. (canceled)

40. The oligomer of claim 4, wherein the oligomer is an oligonucleotide or a peptide nucleic acid (PNA)-oligomer.
